(19) United States
(12) Patent Application Publication
(10) Pub. No.: US 2022/0308039 A1
(43) Pub. Date: Sep. 29, 2022

(54) PHYSICAL QUANTITY ESTIMATING SYSTEM AND PHYSICAL QUANTITY ESTIMATING METHOD

(71) Applicant: Hitachi Metals, Ltd., Tokyo (JP)

(72) Inventors: Chikako Kawamura, Tokyo (JP); Daisuke Shanai, Tokyo (JP); Takehiko Tani, Tokyo (JP)

(21) Appl. No.: 17/698,290

(22) Filed: Mar. 18, 2022

(30) Foreign Application Priority Data
Mar. 24, 2021 (JP) 2021-050044

(51) Int. Cl. G01N 33/44

(52) U.S. Cl. CPC G01N 33/442

(57) ABSTRACT

A physical quantity estimating system for estimating a value of physical quantity for a composite material is provided. The composite material contains two or more materials included in a plurality of different materials as constituent materials. The physical quantity estimating system includes: an approximate function generating unit configured to generate, when a first synthesis characteristic value of a first composite material whose value of physical quantity is unknown is inputted, an approximate function of outputting the value of the physical quantity; a synthesis characteristic value calculating unit configured to calculate the first synthesis characteristic value based on a first blending ratio of constituent materials contained in the first composite material and first related data corresponding to each constituent material; and a physical quantity estimating unit configured to estimate the value of the physical quantity for the first composite material based on the first synthesis characteristic value and the approximate function.

### CROSS-REFERENCE TO RELATED APPLICATION

The present application claims priority from Japanese Patent Application No. 2021-050044 filed on Mar. 24, 2021, the content of which is hereby incorporated by reference into this application.

### TECHNICAL FIELD OF THE INVENTION

The present invention relates to a physical quantity estimating system and a physical quantity estimating method, and relates to a technique effective when applied to a technique for estimating a value of physical quantity according to a blending ratio of a resin composite material, for example.

### BACKGROUND OF THE INVENTION

Japanese Patent Application Publication No. 2020-77257 (hereinafter, referred to as “Patent Document 1”) discloses a technique for estimating a physical property of a material to be designed by utilizing artificial intelligence.

### SUMMARY OF THE INVENTION

In recent years, a composite material in which new performance is imparted to characteristics of a resin itself by compounding plural types of resins or blending agents has been developed. In this regard, in the development of a new composite material, it is necessary to develop a material while adjusting a composition ratio of each composition until the composite material has a desired characteristic. For this reason, the development of the composite material requires enormous costs. Therefore, from the viewpoint of improving the efficiency of development of the composite material, it is desirable that physical quantity for the composite material to be tested at an experimental planning stage can be estimated to an extent. However, for example, there are many types of compounding agents in composite materials for electric wire coating materials. In addition, a value of physical quantity may change significantly depending upon a compounding composition ratio thereof. For this reason, it is difficult to estimate the value of the physical quantity for the composite material. From the above, a technique capable of estimating a value of physical quantity for a composite material with high accuracy is desired.

A physical quantity estimating system according to one embodiment is a physical quantity estimating system for estimating a value of physical quantity for a composite material. The composite material contains two or more materials included in a plurality of different materials as constituent materials. Here, the physical quantity estimating system includes: an approximate function generating unit configured to generate, when a first synthesis characteristic value of a first composite material whose value of physical quantity is unknown is inputted, an approximate function of outputting the value of the physical quantity for the first composite material; a synthesis characteristic value calculating unit configured to calculate the first synthesis characteristic value of the first composite material on a basis of a first blending ratio of constituent materials contained in the first composite material and first related data corresponding to each of the constituent materials contained in the first composite material; and a physical quantity estimating unit configured to estimate the value of the physical quantity for the first composite material on a basis of the first synthesis characteristic value and the approximate function.

Further, a physical quantity estimating system according to one embodiment includes: a related data storage unit configured to store a plurality of related data, a characteristic value of each of a plurality of different materials being associated with a value of physical quantity for the corresponding material in each of the plurality of related data; a blending ratio input unit configured to input a blending ratio of constituent materials of a composite material, the composite material containing two or more materials included in the plurality of different materials as the constituent materials, a value of corresponding physical quantity for the composite material being known; a related data extracting unit configured to extract related data corresponding to each of the constituent materials from the plurality of related data; a synthesis characteristic value calculating unit configured to calculate a synthesis characteristic value of the composite material by executing an operation of synthesizing the characteristic values included in the corresponding related data of the respective constituent materials on a basis of the blending ratio; a synthesis related data generating unit configured to generate synthesis related data in which the synthesis characteristic value is associated with the value of the physical quantity for the composite material; and an approximate function generating unit configured to generate, when a first synthesis characteristic value of a first composite material whose value of the corresponding physical quantity is unknown is inputted, an approximate function of outputting a value of the physical quantity for the first composite material on a basis of the synthesis related data.

Here, the blending ratio input unit is configured to input a first blending ratio of the constituent materials contained in the first composite material; the related data extracting unit is configured to extract first related data corresponding to each of the constituent materials contained in the first composite material from the plurality of related data; and the synthesis characteristic value calculating unit is configured to calculate the first synthesis characteristic value of the first composite material on a basis of the first blending ratio and the first related data.

Further, the physical quantity estimating system further includes: a physical quantity estimating unit configured to estimate a value of the physical quantity corresponding to the first blending ratio on a basis of the first synthesis characteristic value and the approximate function; and an output unit configured to output the value of the physical quantity estimated by the physical quantity estimating unit.

A physical quantity estimating method according to one embodiment is a physical quantity estimating method of estimating a value of physical quantity for a composite material. The composite material contains two or more materials included in a plurality of different materials as constituent materials. Here, the physical quantity estimating method includes: an approximate function generating step configured to generate, when a first synthesis characteristic value of a first composite material whose value of physical quantity is unknown is inputted, an approximate function of outputting the value of the physical quantity for the first composite material; a synthesis characteristic value calculating step configured to calculate the first synthesis characteristic value of the first composite material on a basis of a first blending ratio of constituent materials contained in the first composite material and first related data corresponding to each of the constituent materials contained in the first composite material; and a physical quantity estimating step configured to estimate the value of the physical quantity for the first composite material on a basis of the first synthesis characteristic value and the approximate function.

Further, a physical quantity estimating method according to one embodiment includes: a related data storage step configured to store a plurality of related data in a related data storage unit, a characteristic value of each of a plurality of different materials being associated with a value of physical quantity for the corresponding material in each of the plurality of related data; a blending ratio input step configured to input a blending ratio of constituent materials of a composite material, the composite material containing two or more materials included in the plurality of different materials as the constituent materials, a value of corresponding physical quantity for the composite material being known; a related data extracting step configured to extract related data corresponding to each of the constituent materials from the plurality of related data; a synthesis characteristic value calculating step configured to calculate a synthesis characteristic value of the composite material by executing an operation of synthesizing the characteristic values included in the corresponding related data of the respective constituent materials on the basis of the blending ratio; a synthesis related data generating step configured to generate synthesis related data in which the synthesis characteristic value is associated with the value of the physical quantity for the composite material; and an approximate function generating unit configured to generate, when a first synthesis characteristic value of a first composite material whose value of the corresponding physical quantity is unknown is inputted, an approximate function of outputting a value of the physical quantity for the first composite material on a basis of the synthesis related data.

Here, the blending ratio input step is configured to input a first blending ratio of the constituent materials contained in the first composite material; the related data extracting step is configured to extract first related data corresponding to each of the constituent materials contained in the first composite material from the plurality of related data; and the synthesis characteristic value calculating step is configured to calculate the first synthesis characteristic value of the first composite material on a basis of the first blending ratio and the first related data.

Further, the physical quantity estimating method further includes: a physical quantity estimating step configured to estimate a value of the physical quantity corresponding to the first blending ratio on a basis of the first synthesis characteristic value and the approximate function; and an output step configured to output the value of the physical quantity estimated in the physical quantity estimating step.

According to one embodiment, it is possible to estimate a value of physical quantity for a composite material with high accuracy.

### BRIEF DESCRIPTIONS OF THE DRAWINGS

The foregoing and other objects, features and advantages of the present invention will become more readily apparent from the following detailed description of embodiments of the present invention that proceeds with reference to the appending drawings:

FIG. 1 is a view illustrating one example of a hardware configuration of a physical quantity estimating apparatus;

FIG. 2 is a functional block diagram illustrating functions of the physical quantity estimating apparatus;

FIG. 3 is a view for explaining machine learning for generating an approximate function;

FIG. 4 is a flowchart for explaining a generating operation of the approximate function;

FIG. 5 is a flowchart for explaining an operation of estimating a value of physical quantity for a composite material of an evaluation target;

FIG. 6 is a functional block diagram illustrating an example in which the physical quantity estimating system is configured by a physical quantity estimating apparatus and an approximate function generating apparatus;

FIG. 7 is a functional block diagram illustrating functions of a physical quantity estimating apparatus;

FIG. 8 is a view for explaining machine learning for generating a level approximate function;

FIG. 9 is a view for explaining machine learning for generating an approximate function;

FIG. 10 is a flowchart for explaining a generating operation of a level approximate function;

FIG. 11 is a flowchart for explaining a generating operation of an approximate function;

FIG. 12 is a flowchart for explaining an operation of estimating a value of physical quantity for a composite material of an evaluation target;

FIG. 13A is a table illustrating one example of a composite material used when generating an approximate function, and FIG. 13B is a table illustrating one example of a composite material that becomes an evaluation target; and

FIGS. 14A and 14B are graphs illustrating results of estimating a value of physical quantity for the composite material of the evaluation target illustrated in FIG. 13B using a related technique, and are graphs illustrating results of estimating the physical quantity for the composite material of the evaluation target illustrated in FIG. 13B using a technical idea according to an embodiment.

### DESCRIPTIONS OF EMBODIMENTS

In all of the drawings for explaining an embodiment, in principle, the same reference numeral is assigned to the same member, and repeated explanation thereof will be omitted. Note that hatching may be applied to even a plan view in order to cause the drawings to be understood easily.

The technical idea according to the present embodiment is an idea regarding a physical quantity estimating system for estimating a value of physical quantity corresponding to a blending ratio in a composite material in which plural kinds of resins or blending agents are compounded.

Here, as the composite material, for example, electric wire coating materials containing resins or blending agents can be cited. As the physical quantity, for example, an elongation characteristic of the composite material can be cited.

The resin is a polyolefin such as high-density polyethylene, low-density polyethylene, or an ethylene-acrylic acid copolymer, or an elastomer such as chlorinated polyethylene, for example. On the other hand, as the blending agents, fillers such as talc, calcium carbonate and silica, plasticizers, cross-linking agents, and stabilizers can be cited, for example. However, the types or the number of compositions such as the resins or the blending agents constituting the composite material are not limited.

### Description of Related Technique

First, a related technique regarding a physical quantity estimating system for estimating a value of physical quantity corresponding to a blending ratio will be described. The “related technique” referred to in the present specification is not a known technique, but is a technique that has problems found by the inventors of the present application and that is a premise of the present invention.

For example, as the physical quantity estimating system, it is conceivable a related technique of estimating a value of physical quantity of a composite material on the basis of an approximate function that output, when a material name of each of constituent materials constituting the composite material and a blending ratio of the constituent materials are inputted, the value of the physical quantity of this composite material. In this related technique, for example, data in which the material name of each of the constituent materials, the blending ratio of the constituent materials, and a value of physical quantity corresponding to this lending ratio are known are used as teacher data, and an approximate function in which an input is the material names and the blending ratio and an output is the value of the physical quantity is generated.

However, in the related technique, an estimate target of the value of the physical quantity is limited to the constituent materials contained in the teacher data used for generating the approximate function. Namely, in a case where any constituent material that has not been used for generating the approximate function is contained in the composite material that becomes an evaluation target, estimation accuracy of the value of the physical quantity of this composite material is lowered. This is because, in the related technique, since the material names of the constituent materials are used as an input parameter of the approximate function, synthesis of input parameters cannot be executed. This point will be explained simply.

For example, it is assumed that data in which a value of physical quantity of “100” is associates with a material name of “high-density polyethylene” and data in which a value of physical quantity of “200” is associated with a material name of “low-density polyethylene” are used as teacher data to generate an approximate function of the related technique.

In this case, for example, it is conceivable that the approximate function generated by the related technique is used to estimate a value of physical quantity for a composite material in which the “high-density polyethylene” and the “low-density polyethylene” are contained as constituent materials of the composite material and a blending ratio of the constituent materials is 50:50.

First, in the related technique, when it is considered that in order to obtain the input parameters for the composite material, the input parameters for the constituent materials constituting the composite material are synthesized, it becomes an operation of “high-density polyethylene”×0.5+“low-density polyethylene”×0.5 to “material name”×“numerical value”. Therefore, the operation of synthesizing the input parameters itself does not make sense.

However, the data in which the value of the physical quantity of “100” is associated with the material name of the “high-density polyethylene” and the data in which the value of the physical quantity of “200” is associated with the material name of the “low-density polyethylene” are used as the teacher data. For this reason, in this case, it is assumed that it is possible to estimate that the value of the physical quantity for the composite material is “100”×0.5+“200”×0.5=“150” in the approximate function generated by the related technique without executing the operation of the synthesis of the input parameters. Namely, in the related technique, it is conceivable that it is possible to estimate the value of the physical quantity for the composite material containing the “high-density polyethylene” and the “low-density polyethylene” used for the teacher data with high accuracy.

On the other hand, for example, it is considered that an approximate function generated by the related technique is used to estimate a value of physical quantity for a composite material that contains “polyolefin” and “high-density polyethylene” as constituent materials of the composite material and in which a blending ratio of the constituent materials is 70:30.

In this case, first, it is also considered that in order to obtain input parameters for the composite material, the input parameters for the constituent materials constituting the composite material are synthesized. It becomes “polyolefin”×0.7+“high-density polyethylene”×0.3 to become an operation of “material name”×“numerical value”. Therefore, the operation itself of synthesis of the input parameters do not make sense.

Moreover, in this case, the “polyolefin” that is not contained in the teacher data is not included as the constituent materials of the composite material. As a result, in the approximate function generated by the related technique, it is difficult to grasp a value of physical quantity for the “polyolefin”. Therefore, the value of the physical quantity for the composite material becomes “???”×0.7+“100”×0.3. Thus, it becomes difficult to estimate the value of the physical quantity for the composite material containing the “polyolefin” and the “high-density polyethylene” with high accuracy.

This means that in the approximate function using the material names as the input parameters, a synthesis operation of the input parameters does not make sense, and thus, estimation accuracy of the value of the physical quantity for the composite material containing a constituent material, which is not used for the teacher data, is lowered.

From the above, there is room for improvement in the related technique from the viewpoint of accurately estimating the value of the physical quantity for the composite material containing the constituent material that has not used for generating the approximate function.

Therefore, in the present embodiment, some measures are taken for the room for improvement existing the related technique. Hereinafter, the technical idea according to the present embodiment to which such measures are applied will be described.

### Fundamental Thought According to Embodiment

First, the inventors of the present application focused on the essence of a problem in that as a result of using input parameters of material names, by which it is difficult to execute a synthesis operation, in the related technique, estimation accuracy of a value of physical quantity for a composite material containing a constituent material that is not used for teacher data is reduced. Then, the inventors of the present application acquired the finding that the estimation accuracy of the value of the physical quantity for the composite material containing the constituent material that is not used for the teacher data can be improved by using an input parameter regarding a constituent material by which it is easy to execute a synthesis operation, for example.

In this regard, a fundamental thought is an idea that if parameters that can be expressed by numerical values are used as input parameters regarding constituent materials, it becomes possible to execute the synthesis operation, and this makes it possible to improve estimation accuracy of a value of physical quantity for a composite material containing a constituent material that is not used for teacher data. Hereinafter, this point will specifically be described.

For example, it is assumed that data in which a value of physical quantity of “100” is associated with an input parameter of “50” and data in which a value of physical quantity of “150” is associated with an input parameter of “100” are used for teacher data to generate an approximate function.

In this regard, it is first thought that a value of physical quantity for a composite material that contains a constituent material whose input parameter is “50” and a constituent material whose input parameter is “100” as constituent materials of the composite material and in which a blending ratio of the constituent materials is 50:50 is estimated by using the approximate function described above, for example. In this case, in order to obtain an input parameter for the composite material, when it is thought to synthesize the input parameters for the constituent materials constituting the composite material, it becomes “50”×0.5+“100”×0.5=“75”. Because of an operation of “numerical value”×“numerical value”, it is possible to execute an operation of synthesis of the input parameters easily. This makes it possible to obtain the input parameter for the composite material as “75”. Thus, by inputting this input parameter of “75” into the approximate function according to the fundamental thought, it is possible to estimate the value of the physical quantity for the composite material. Therefore, according to the fundamental thought, it can be seen that it is also possible to estimate the value of the physical quantity for the composite material containing the constituent materials used for the teacher data with high accuracy.

Subsequently, it is thought that a value of physical quantity for a composite material that contains a constituent material whose input parameter is “50” and a constituent material whose input parameter is “75” as constituent materials of the composite material and in which a blending ratio of the constituent materials is 50:50 is estimated by using the approximate function described above, for example.

In this case, the constituent material whose input parameter is “75” and that is not included for teacher data is contained as the constituent materials of the composite material. However, in the fundamental thought, the parameters expressed by numerical values are used as the input parameters. For this reason, in the fundamental thought, in order to obtain an input parameter for the composite material, it is possible to synthesize the input parameters for the constituent materials constituting the composite material. Specifically, when it is thought to synthesize the input parameters for the constituent materials constituting the composite material, it becomes “50”×0.5+“75”×0.5=“62.5”. Because of an operation of “numerical value”×“numerical value”, it is possible to execute an operation of synthesis of the input parameters easily. This makes it possible to obtain the input parameter for the composite material as “62.5”. Thus, by inputting this input parameter of “62.5” into the approximate function according to the fundamental thought, it is possible to estimate the value of the physical quantity for the composite material. Therefore, according to the fundamental thought, it can be seen that it is also possible to estimate a value of physical quantity for a composite material containing anew constituent material that is not used for teacher data with high accuracy. This is a result of using input parameters that can be synthesized easily, that is, parameters that can be expressed by numerical values as input parameters regarding constituent materials in the fundamental thought. Thus, the essence of the fundamental thought is to use numerical values for which a synthesis operation is possible as the input parameters regarding the constituent materials.

Here, as the parameters that can be expressed by numerical values as the input parameters regarding the constituent materials, the inventors of the present application focus on a characteristic value of a constituent material.

Namely, the fundamental thought according to the present embodiment is an idea to estimate a value of physical quantity for a composite material by using an approximate function generated on the basis of characteristic values (numerical values) of constituent materials constituting the composite material and a blending ratio of the constituent materials.

According to this fundamental thought, since the approximate function generated on the basis of the characteristic values of the constituent materials and the blending ratio of the constituent materials is used, it is possible to obtain effects as follows.

For example, in an approximate function generated on the basis of material names of constituent materials and a blending ratio of the constituent materials as in the related technique, input parameters are material names of constituent materials and a blending ratio of the constituent materials. For this reason, in a case where a new constituent material that is not used to generate the approximate function is contained in a composite material that becomes an evaluation target, the concept of a synthesis operation of the material names of the constituent materials used when the approximate function is generated and a material name of the new constituent material does not make sense. Thus, estimation accuracy of a value of physical quantity for this composite material is reduced. Namely, the approximate function generated by the related technique narrows an application range of a composite material in which it is possible to estimate the value of the physical quantity with high accuracy.

In particular, in the related technique, even though a material name (new material name) of a new constituent material that was not used to generate an approximate function is known, a value of physical quantity for a composite material that becomes an evaluation target cannot be estimated with high accuracy by the approximate function generated by the related technique unless a value of physical quantity for the new constituent material is known. In other words, a value of physical quantity for only a composite material containing only constituent materials used for teacher data can be estimated with high accuracy by the approximate function generated by the related technique.

On the other hand, in the fundamental thought according to the present embodiment, an approximate function is generated on the basis of characteristic values of constituent materials and a blending ratio of the constituent materials. In this case, even though a new constituent material that was not used to generate an approximate function is contained in a composite material that becomes an evaluation target, it is possible to estimate a value of physical quantity for the composite material with high accuracy so long as a characteristic value corresponding to this new constituent materials is known. This is because the characteristic values of the constituent materials can be expressed by numerical values, and this makes it possible to execute a synthesis operation.

As described above, an application range of the approximate function generated by the fundamental thought is wider than the application range of the approximate function generated by the related technique, and has great technical significance in that a value of physical quantity for a composite material can be estimated with high accuracy even in a case where a new constituent material that was not used for teacher data is contained in the composite material that becomes an evaluation target. Namely, the application range of the approximate function generated by the related technique is limited to a range of the teacher data. However, it can be said that the fundamental thought is an excellent technical idea in that the application range of the approximate function generated by the fundamental thought is not limited to the range of the teacher data. For example, according to the fundamental thought, it is possible to estimate a value of physical quantity for a composite material containing a new constituent material, which was not considered at the time of generation of an approximate function, with high accuracy by accumulating characteristic values for new constituent materials that were not used to generate the approximate function as a database. Moreover, by using the approximate function according to the fundamental thought, even though a new constituent material is not stored in the database, it is possible to estimate a value of physical quantity for a composite material containing the new constituent material with high accuracy so long as a characteristic value of this new constituent material can be obtained by any means. In view of this point, the application range of the approximate function generated by the fundamental thought is large.

Here, the “characteristic value” means thermal characteristics, mechanical characteristics, physical properties, and the like, for example. For example, the thermal characteristics include heat of fusion, a melt flow rate, and the like. Further, the physical properties include specific gravity. On the other hand, an elongation characteristic and the like are assumed as the “physical quantity”.

In the present specification, the “characteristic value” and the “value of physical quantity” are used so as to be clearly distinguished. Specifically, the “characteristic value” is a parameter used for generation of an approximate function and an input of an approximate function. On the other hand, the “value of physical quantity” is a value to be outputted from an approximate function, and is a target value to be estimated by a physical quantity estimating system according to the present embodiment.

Hereinafter, an example in which a physical quantity estimating system embodying the fundamental thought is configured by a single computer will mainly be described. However, the physical quantity estimating system according to the present embodiment can be realized by a distributed system consisting of a plurality of computers.

### Configuration of Physical Quantity Estimating Apparatus

### <<Hardware Configuration>>

First, a hardware configuration of a physical quantity estimating apparatus according to the present embodiment will be described.

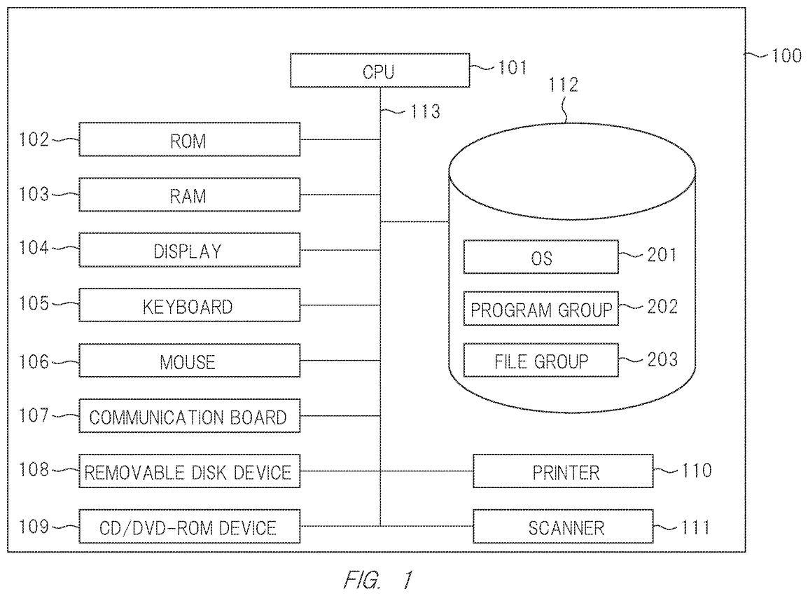

FIG. 1 is a view illustrating one example of a hardware configuration of a physical quantity estimating apparatus 100 according to the present embodiment. Note that the configuration illustrated in FIG. 1 is merely one example of the hardware configuration of the physical quantity estimating apparatus 100. The hardware configuration of the physical quantity estimating apparatus 100 is not limited to the configuration illustrated in FIG. 1, and may be any other configuration.

In FIG. 1, the physical quantity estimating apparatus 100 includes a CPU (Central Processing Unit) 101 configured to execute programs. This CPU 101 is electrically connected to a ROM (Read Only Memory) 102, a RAM (Random Access Memory) 103, and a hard disk device 112 via a bus 113, for example, and is configured to control these hardware devices.

Further, the CPU 101 is also connected to an input device and an output device via the bus 113. As examples of the input device, a keyboard 105, a mouse 106, a communication board 107, a scanner 111, and the like can be cited. On the other hand, as examples of the output device, a display 104, the communication board 107, a printer 110, and the like can be cited. Moreover, the CPU 101 may also be connected to a removable disk device 108 or a CD/DVD-ROM device 109, for example.

The physical quantity estimating apparatus 100 may be connected to a network, for example. In a case where the physical quantity estimating apparatus 100 is connected to another external apparatus via the network, for example, the communication board 107 constituting a part of the physical quantity estimating apparatus 100 is connected to a LAN (local area network), a WAN (wide area network), or the Internet.

The RAM 103 is one example of a volatile memory. The recording media such as the ROM 102, the removable disk device 108, the CD/DVD-ROM device 109, and the hard disk device 112 are examples of non-volatile memory. A storage device of the physical quantity estimating apparatus 100 is configured by these volatile memories and non-volatile memories.

An operating system (OS) 201, a program group 202, and a file group 203 are stored in the hard disk device 112, for example. The CPU 101 executes programs included in the program group 202 while using the operating system 201. Further, at least a part of a program of the operating system 201 and an application program to be executed by the CPU 101 is temporarily stored in the RAM 103, and various kinds of data necessary for processes by the CPU 101 are also stored therein.

A BIOS (Basic Input Output System) program is stored in the ROM 102, and a boot program is stored in the hard disk device 112. When the physical quantity estimating apparatus 100 is started, the BIOS program stored in the ROM 102 and the boot program stored in the hard disk device 112 are executed, and the operating system 201 is started by the BIOS program and the boot program.

Programs for realizing functions of the physical quantity estimating apparatus 100 are stored in the program group 202, and these programs are read out and executed by the CPU 101. Further, information, data, signal values, variable values, and parameters, which indicate results of the processes by the CPU 101, are stored in the file group 203 as each item of the file.

The file is stored in the recording media such as the hard disk device 112 or the memories. The information, the data, the signal values, the variable values, and the parameters, which are stored in the recording media such as the hard disk device 112 or the memories, are read out to a main memory or a cache memory by the CPU 101, and are used for the operation of the CPU 101 represented by extraction, search, reference, comparison, calculation, processing, editing, output, printing, and display. For example, during the operation of the CPU 101 described above, the information, the data, the signal values, the variable values, and the parameters are temporarily stored in the main memory, a register, the cache memory, the buffer memory, and the like.

The functions of the physical quantity estimating apparatus 100 may be realized by firmware stored in the ROM 102. Alternatively, the functions may be realized by only software, only hardware represented by elements, devices, boards, and wirings, a combination of software and hardware, or a combination of them and the firmware. The firmware and the software are stored as programs in the recording media represented by the hard disk device 112, a removable disk, a CD-ROM, a DVD-ROM, or the like. The programs are read out and executed by the CPU 101. Namely, the programs cause a computer to serve as the physical quantity estimating apparatus 100.

As described above, the physical quantity estimating apparatus 100 is a computer that includes the CPU 101 as a processing device, the hard disk device 112 and the memories as storage devices, the keyboard 105, the mouse 106, and the communication board 107 as the input devices, and the display 104, the printer 110, and the communication board 107 as the output devices. The functions of the physical quantity estimating apparatus 100 are realized by using the processing device, the storage devices, the input devices, and the output devices.

### <<Functional Block Configuration>>

Next, a functional block configuration of the physical quantity estimating apparatus 100 will be described.

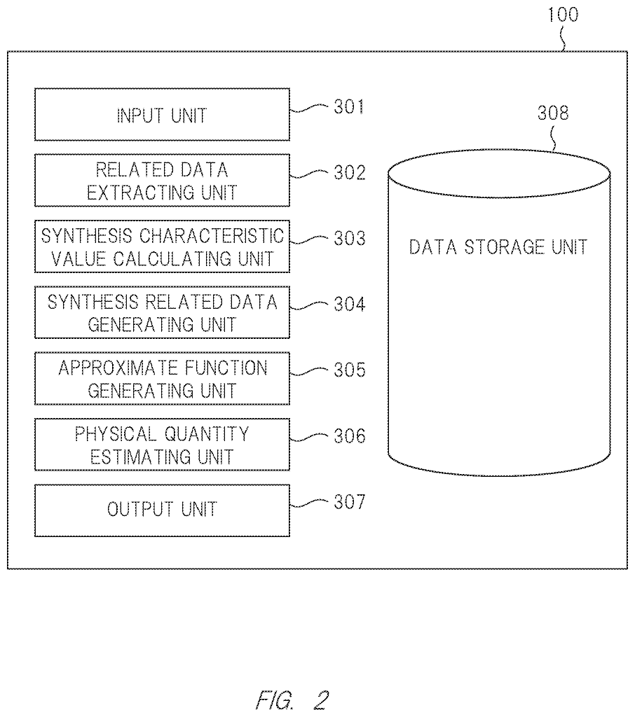

FIG. 2 is a functional block diagram illustrating functions of the physical quantity estimating apparatus 100.

The physical quantity estimating apparatus 100 includes an input unit 301, a related data extracting unit 302, a synthesis characteristic value calculating unit 303, a synthesis related data generating unit 304, an approximate function generating unit 305, a physical quantity estimating unit 306, an output unit 307, and a data storage unit 308.

The input unit 301 is configured to input related data. Here, the “related data” mean data in which a characteristic value of each of a plurality of different materials is associated with a value of physical quantity for the corresponding material. The related data are data in which a material name, a characteristic value, and a value of physical quantity are associated with each other, for example. The related data inputted by the input unit 301 are stored in the data storage unit 308. This data storage unit 308 serves as a database for storing a plurality of related data.

Further, the input unit 301 is configured to input a blending ratio of constituent materials of a composite material. The composite material contains two or more materials included in the plurality of different materials as the constituent materials. A value of corresponding physical quantity for composite material is known. The blending ratio inputted by the input unit 301 is also stored in the data storage unit 308. Moreover, the input unit 301 is also configured to input a blending ratio of constituent materials contained in a composite material whose value of corresponding physical quantity is unknown in addition to the blending ratio of the constituent materials contained in the composite material whose value of the corresponding physical quantity is known. This blending ratio is also stored in the data storage unit 308.

The related data extracting unit 302 is configured to extract related data corresponding to each of the constituent materials contained in the composite material from the plurality of related data stored in the data storage unit 308. For example, in a case where the constituent materials contained in the composite material are “polyolefin” and “polyethylene”, the related data extracting unit 302 is configured to extract related data corresponding to “polyolefin” and related data corresponding to “polyethylene” from the plurality of related data.

The synthesis characteristic value calculating unit 303 is configured to calculate a synthesis characteristic value of the composite material by executing an operation of synthesizing the characteristic values included in the corresponding related data of the respective constituent materials constituting the composite material on the basis of the blending ratio inputted by the input unit 301.

For example, as constituent materials of a composite material, it is considered a composite material that contains a constituent material whose characteristic value is “50” and a constituent material whose characteristic value is “75” and in which a blending ratio of the constituent materials is 50:50. In this case, the synthesis characteristic value calculating unit 303 executes a synthesis operation of “50”×0.5+“75”×0.5=“62.5” to calculate a synthesis characteristic value of “62.5”.

The synthesis characteristic values include synthesis heat of fusion of the composite material, a synthesis melt flow rate of the composite material, and the like, for example.

The synthesis related data generating unit 304 is configured to generate synthesis related data in which the synthesis characteristic value calculated by the synthesis characteristic value calculating unit 303 is associated with the value of the physical quantity for the composite material. This generation of the synthesis related data is executed for the composite material inputted by the input unit 301 and whose corresponding physical quantity is known. For example, in a case where the value of the physical quantity for the composite material in the example described above is “150”, the synthesis related data generating unit 304 generates synthesis related data in which the synthesis characteristic value of “62.5” is associated with the value of the physical quantity of “150”. The generated synthesis related data are stored in the data storage unit 308.

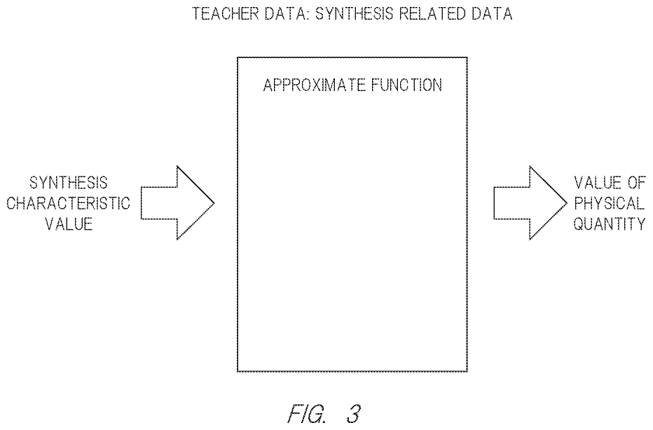

The approximate function generating unit 305 has a function of generating an approximate function on the basis of the synthesis related data generated by the synthesis related data generating unit 304. Namely, the approximate function generating unit 305 is configured to generate an approximate function of associating the synthesis characteristic value with the value of the physical quantity. Specifically, as illustrated in FIG. 3, the approximate function generating unit 305 is configured to generate an approximate function in which the synthesis related data are used as teacher data, an input is the synthesis characteristic value, and an output is the value of the physical quantity.

Here, the “approximate function” is defined as a function of outputting, when a synthesis characteristic value is inputted, a value of physical quantity according to this synthesis characteristic value. Namely, the “approximate function” is defined as a function of outputting, in a case where a synthesis characteristic value of a composite material whose correspondence with a value of physical quantity is unknown is inputted, a value of physical quantity that is presumed to be realized by this synthesis characteristic value. Thus, the approximate function can be said to be a function used to estimate a value of physical quantity for a composite material whose correspondence with the value of the physical quantity is unknown.

The physical quantity estimating unit 306 has a function of estimating a value of physical quantity corresponding to a first composite material on the basis of a first synthesis characteristic value calculated by the synthesis characteristic value calculating unit 303 based on a first blending ratio of the first composite material and first related data and the approximate function generated by the approximate function generating unit 305.

Note that the “first composite material” is a composite material that contains two or more materials included in the plurality of different materials as constituent materials, and indicates a composite material whose corresponding value of physical quantity is unknown and which becomes an evaluation target. Here, a blending ratio of the first composite material is called a “first blending ratio”, and a synthesis characteristic value of the first composite material is called a “first synthesis characteristic value”.

Further, among the related data stored in the data storage unit 308, related data corresponding to each of the constituent materials contained in first composite material are called as “first related data”.

For example, the first blending ratio is inputted into the physical quantity estimating apparatus 100 from the input unit 301, and the first related data are extracted by the related data extracting unit 302.

The output unit 307 is configured to output the value of the physical quantity estimated by the physical quantity estimating unit 306.

The physical quantity estimating apparatus 100 is configured in this manner.

The constituent materials of the composite material include a plurality of different types of resins, for example. However, other constituent materials may be included. For example, additives, antioxidants, cross-linking aids, or the like may be contained as the constituent materials of the composite material. Further, as one example of the resins, a crosslinked resin can be cited. Since an additional function is added to the physical quantity estimating apparatus 100 depending upon concrete constituent materials of a composite material, this point will be described below.

<<<Case where Composite Material Contains Additives>>>

In a case where additives are contained in a composite material, the synthesis characteristic value calculating unit 303 is further configured to calculate an average inter-filler distance of the additives or a volume fraction of the additives on the basis of a characteristic value of the additives in addition to the functions described above. The average inter-filler distance of the additives or the volume fraction of the additives is included in the synthesis characteristic value calculated by the synthesis characteristic value calculating unit 303.

Note that an average inter-particle distance represented by the average inter-filler distance is calculated from an average particle size D50 using a theoretical formula, for example. Further, the volume fraction is calculated from specific gravity of the compounded material.

<<<Case where Composite Material Contains Antioxidants and Cross-Linking Aids>>>

In a case where antioxidants and cross-linking aids are contained in a composite material, the synthesis characteristic value calculating unit 303 is further configured to calculate a reaction mole number of a primary reactive group of the antioxidants, a reaction mole number of a secondary reactive group of the antioxidants and a reaction mole number of the cross-linking aids on the basis of a characteristic value of the antioxidants and a characteristic value of the cross-linking aids in addition to the functions described above. The reaction mole number of the primary reactive group of the antioxidants, the reaction mole number of the secondary reactive group of the antioxidants and the reaction mole number of the cross-linking aids are included in the synthesis characteristic value calculated by the synthesis characteristic value calculating unit 303.

<<<Case where Composite Material Contains Crosslinked Resin>>>

In a case where a crosslinked resin is contained in a composite material, the input unit 301 is further configured to also input a radiation dose for crosslinking the resin. Further, the approximate function generating unit 305 is configured to generate an approximate function on the basis of the synthesis related data and the radiation dose. The approximate function in this case is generated as a function in which an input is a synthesis characteristic value and a radiation dose and an output is a value of physical quantity. Further, the physical quantity estimating unit 306 is configured to estimate a value of physical quantity for a first composite material on the basis of a first synthesis characteristic value, a first radiation dose, and an approximate function. Here, the “first radiation dose” indicates a radiation dose applied to the first composite material.

### Operation of Physical Quantity Estimating Apparatus

The physical quantity estimating apparatus 100 according to the present embodiment is configured as described above, and an operation thereof will be described below. As the operation of the physical quantity estimating apparatus 100, there are a “generating operation of an approximate function” and an “estimating operation of a value of physical quantity corresponding to a composite material of an evaluation target”. For this reason, these operations will be described below.

### <<Generating Operation of Approximate Function>>

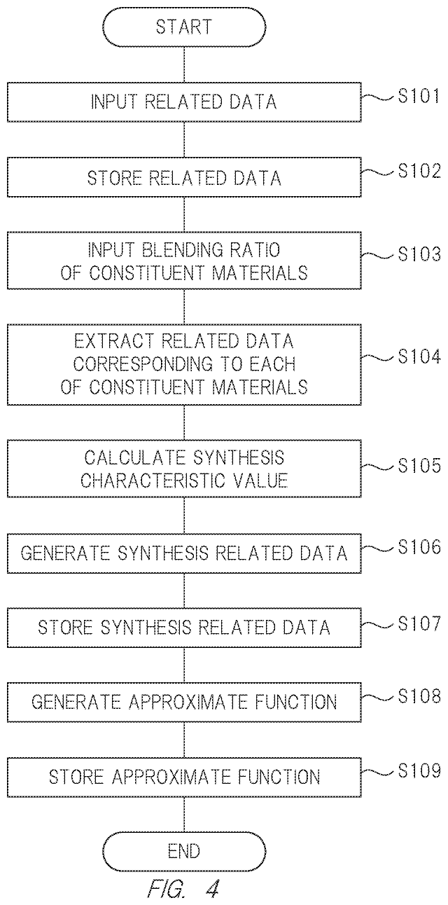

FIG. 4 is a flowchart for explaining a generating operation of an approximate function.

In FIG. 4, the input unit 301 first inputs a plurality of related data in which a characteristic value of each of a plurality of different materials is associated with a value of physical quantity for the corresponding material (S101). The plurality of related data inputted by the input unit 301 is stored in the data storage unit 308 (S102).

Next, the input unit 301 inputs a blending ratio of constituent materials contained in a composite material that contains two or more materials as the constituent materials and whose value of the corresponding physical quantity is known (S103).

The related data extracting unit 302 then extracts related data corresponding to each of the constituent materials contained in the composite material from the plurality of related data stored in the data storage unit 308 (S104). Subsequently, the synthesis characteristic value calculating unit 303 calculates a synthesis characteristic value of the composite material by executing an operation of synthesizing the characteristic values contained in the respective related data extracted by the related data extracting unit 302 on the basis of the blending ratio inputted by the input unit 301 (S105).

The synthesis related data generating unit 304 then generates synthesis related data in which the synthesis characteristic value calculated by the synthesis characteristic value calculating unit 303 is associated with the value of the physical quantity for the composite material (S106). The synthesis related data generated by the synthesis related data generating unit 304 are then stored in the data storage unit 308 (S107).

Next, the approximate function generating unit 305 generates an approximate function on the basis of the synthesis related data generated by the synthesis related data generating unit 304 (S108). Specifically, the approximate function generating unit 305 generates the approximate function in which the synthesis related data are used as teacher data, an input is the synthesis characteristic value, and an output is the value of the physical quantity (see FIG. 3).

The approximate function generated by the approximate function generating unit 305 is then stored in the data storage unit 308 (S109). The generating operation of the approximate function is executed in this manner.

### <<Estimating Operation of Value of Physical Quantity for Composite Material of Evaluation Target>>

Next, an operation for estimating a value of physical quantity for a composite material of an evaluation target will be described.

FIG. 5 is a flowchart for explaining an operation of estimating a value of physical quantity for a composite material of an evaluation target. Here, it is assumed that an approximate function has already been stored in the data storage unit 308.

In FIG. 5, the input unit 301 first inputs a first blending ratio of constituent materials contained in a first composite material that becomes an evaluation target. Correspondence of the first composite material with a value of physical quantity is unknown (S201).

Next, the related data extracting unit 302 extracts first related data of each of the constituent materials contained in the first composite material from a plurality of related data stored in the data storage unit 308 (S202).

Subsequently, the synthesis characteristic value calculating unit 303 calculates a first synthesis characteristic value of the first composite material by executing an operation of synthesizing a characteristic value contained in the first related data extracted by the related data extracting unit 302 on the basis of the first blending ratio inputted by the input unit 301 (S203).

The physical quantity estimating unit 306 then estimates the value of the physical quantity for the first composite material by inputting the first synthesis characteristic value calculated by the synthesis characteristic value calculating unit 303 into the approximate function (S204). The output unit 307 then outputs the value of the physical quantity estimated by the physical quantity estimating unit 306 (S205). As described above, according to the physical quantity estimating apparatus 100, it is possible to output the value of the physical quantity that is highly likely to be realized for the first composite material whose correspondence with the value of the physical quantity is unknown and that becomes the evaluation target.

### Modification Example

In the embodiment, as illustrated in FIG. 2, the example in which a physical quantity estimating system configured to estimate a value of physical quantity for a composite material is configured by the single physical quantity estimating apparatus 100 has been described. However, the physical quantity estimating system is not limited to this configuration, and for example, it can also be configured by a distributed system.

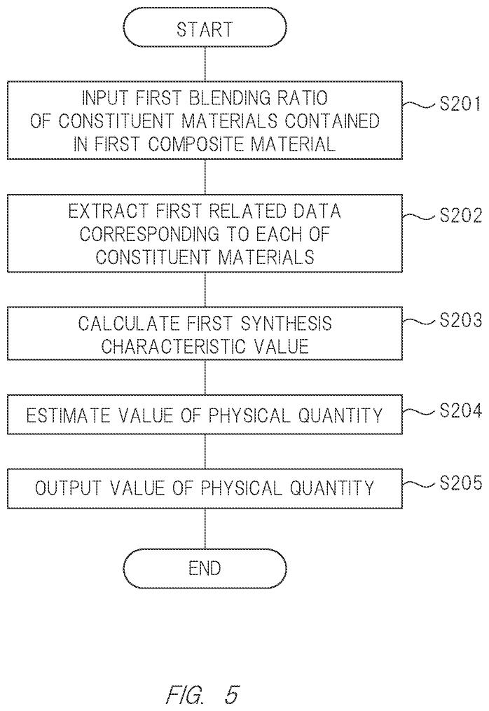

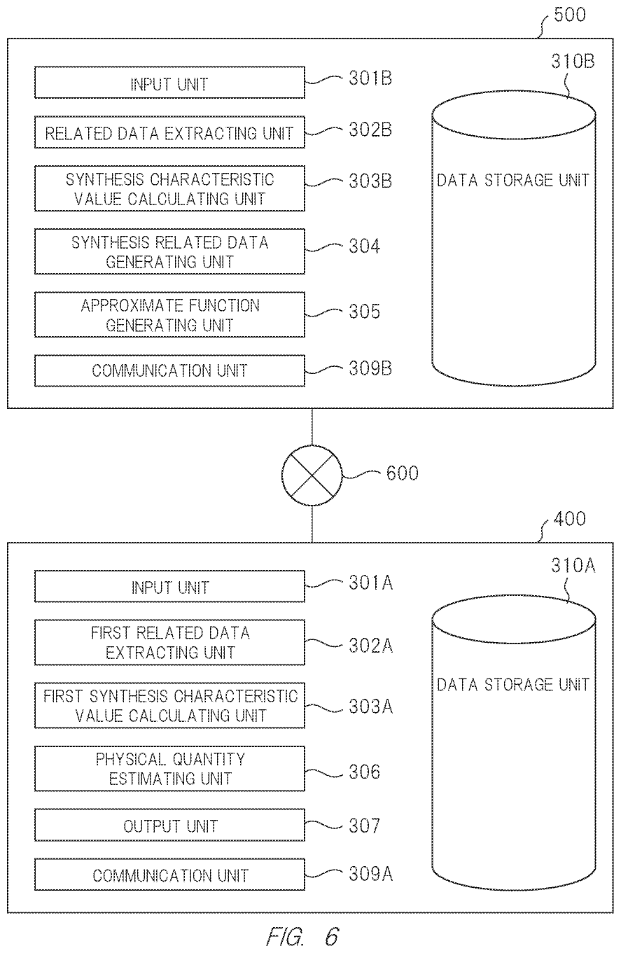

FIG. 6 is a functional block diagram illustrating an example in which a physical quantity estimating system is configured by a physical quantity estimating apparatus and an approximate function generating apparatus.

As illustrated in FIG. 6, a physical quantity estimating system is configured by a physical quantity estimating apparatus 400 and an approximate function generating apparatus 500. For example, the physical quantity estimating apparatus 400 and the approximate function generating apparatus 500 are connected to each other via a network 600.

The physical quantity estimating apparatus 400 includes an input unit 301A, a first related data extracting unit 302A, a first synthesis characteristic value calculating unit 303A, a physical quantity estimating unit 306, an output unit 307, a communication unit 309A, and a data storage unit 310A.

The approximate function generating apparatus 500 includes an input unit 301B, a related data extracting unit 302B, a synthesis characteristic value calculating unit 303B, a synthesis related data generating unit 304, an approximate function generating unit 305, a communication unit 309B, and a data storage unit 310B.

The physical quantity estimating apparatus 400 and the approximate function generating apparatus 500 configured in this manner are configured so that data can be transmitted and received by the communication unit 309A and the communication unit 309B via the network 600. Further, the approximate function generating apparatus 500 executes the “generating operation of an approximate function” described above to generate an approximate function.

On the other hand, the approximate function generated by the approximate function generating apparatus 500 is inputted into the physical quantity estimating apparatus 400 from the approximate function generating apparatus 500, and is stored in the data storage unit 310A.

The physical quantity estimating apparatus 400 then executes the “estimating operation of the value of the physical quantity for the composite material of the evaluation target” described above on the basis of the approximate function stored in the data storage unit 310A.

As described above, it is also possible to construct the physical quantity estimating system according to the present embodiment by the distributed system that includes the physical quantity estimating apparatus 400 and the approximate function generating apparatus 500.

### Further Measures for Improving Estimation Accuracy

### <<Room for Improvement>>

For example, the inventors of the present application newly found that if a numerical range of a value of physical quantity is wide in data in which a synthesis characteristic value of a composite material and a value of physical quantity corresponding to this synthesis characteristic value are known, it is difficult for an approximate function generated using these data as teacher data to estimate a value of physical quantity, which is highly likely to be realized by a composite material whose correspondence with the value of the physical quantity is unknown, with high accuracy. Namely, the inventors of the present application newly found that in a case where a “single approximate function” is generated by using data in which a numerical range of a value of physical quantity is wide as teacher data, it is difficult to estimate physical quantity with high accuracy by the “single approximate function” generated in this manner.

Namely, there is room for improvement in the technique of estimating a value of physical quantity using a “single approximate function” from the viewpoint of estimating a value of physical quantity for a composite material with high accuracy.

Therefore, in the present embodiment, the measures are taken for the room for improvement described above. Hereinafter, the technical idea according to the present embodiment to which the measures are applied will be described.

### <<Technical Idea According to Embodiment>>

The technical idea according to the present embodiment is an idea as follows. Namely, in synthesis related data in which a synthesis characteristic value and a value of physical quantity corresponding to this synthesis characteristic value are known, a numerical range of the value of the physical quantity is divided into a plurality of ranges, and an approximate function peculiar to each of the plurality of ranges is generated by using synthesis related data belonging to the corresponding range of the plurality of divided ranges as teacher data. Namely, the technical idea according to the present embodiment is an idea to generate a different approximate function for each of a plurality of ranges. Namely, according to the technical idea, a “single approximate function” is not generated, but “a plurality of approximate functions” is generated.

According to this technical idea, synthesis related data in a narrow numerical range belonging to each of a plurality of ranges are used as teacher data. Therefore, the approximate functions generated using these synthesis related data as the teacher data allow a value of physical quantity, which is highly likely to be realized by a composite material whose correspondence with the value of the physical quantity is unknown, to be estimated with high accuracy.

In order to estimate a value of physical quantity, which is highly likely to be realized by a composite material whose correspondence with the value of the physical quantity is unknown, by this technical idea, it is necessary to accurately estimate which range of a plurality of ranges the value of the physical quantity for the composite material belongs to. This is because if it is impossible to accurately estimate which range of a plurality of ranges the value of the physical quantity for the composite material belongs to, an approximate function in a range different from the actual one will be used, and as a result, it becomes impossible to estimate the value of the physical quantity with high accuracy.

Therefore, in embodying the technical idea, it becomes important to accurately estimate which range of the plurality of ranges the value of the physical quantity for the composite material belongs to. Taking this point into consideration, the measures to embody the technical idea is taken for it.

### <<Functional Block Configuration>>

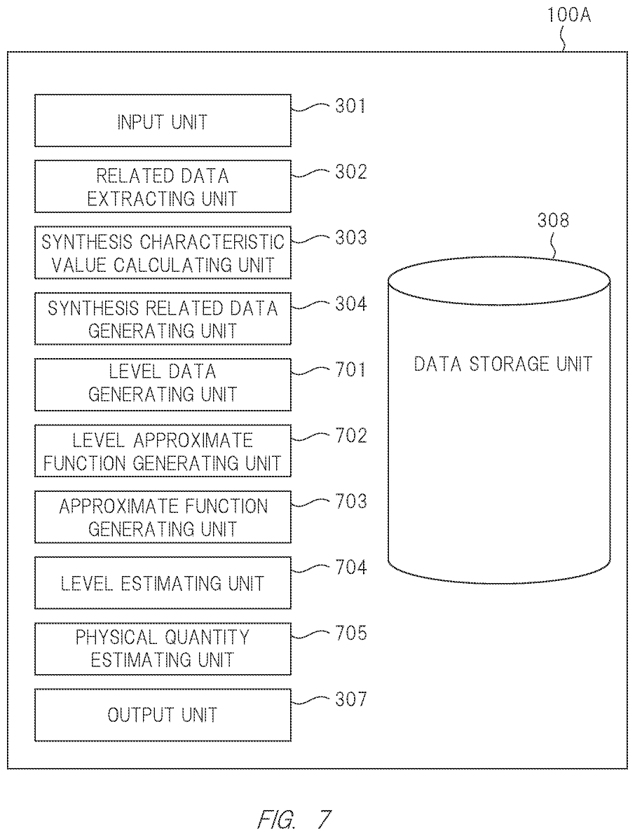

FIG. 7 is a functional block diagram illustrating functions of a physical quantity estimating apparatus.

A functional configuration of a physical quantity estimating apparatus 100A illustrated in FIG. 7 has a substantially similar functional configuration to the functions of the physical quantity estimating apparatus 100 illustrated in FIG. 2. Therefore, differences will mainly be described.

A level data generating unit 701 is configured to generate level data from synthesis related data. Here, the “level data” mean data in which a level indicating which numerical range a value of physical quantity of the synthesis related data belongs to is added to the synthesis related data.

Specifically, the “level data” are defined as data in which a level of a plurality of levels set in advance to which a value of physical quantity belongs is added to synthesis related data and a synthesis characteristic value, the value of the physical quantity, and the level are thus associated with each other.

For example, it is assumed that as synthesis related data, there are first synthesis related data in which a synthesis characteristic value is “10” and a value of physical quantity is “100”, second synthesis related data in which the synthesis characteristic value is “20” and the value of the physical quantity is “500”, and third synthesis related data in which the synthesis characteristic value is “50” and the value of the physical quantity is “200”. At this time, it is assumed that in the level data generating unit 701, for example, a threshold value of the value of physical quantity is set to “300”, and a level is determined by associating data with the value of the physical quantity of “300” or less with a first level and associating data with the value of the physical quantity of over “300” with a second level.

In this case, the first synthesis related data belong to the first level because the value of the physical quantity thereof is “100” and it is “300” or less. The level data generating unit 701 associates the first synthesis related data with the first level, and generates first level data in which the synthesis characteristic value is “10”, the value of the physical quantity is “100”, and the level is the “first level”.

Further, the second synthesis related data belong to the second level because the value of the physical quantity thereof is “500” and it is larger than “300”. The level data generating unit 701 associates the second synthesis related data with the second level, and generates second level data in which the synthesis characteristic value is “20”, the value of the physical quantity is “500”, and the level is the “second level”.

Moreover, the third synthesis related data belong to the first level because the value of the physical quantity thereof is “200” and it is “300” or less. The level data generating unit 701 associates the third synthesis related data with the first level, and generates third level data in which the synthesis characteristic value is “50”, the value of the physical quantity is “200”, and the level is the “first level”.

As a result, the first level data and the third level data become data belonging to the first level, and the second level data become data belonging to the second level.

The level data generated in this manner by the level data generating unit 701 are classified for each level and stored in the data storage unit 308, for example.

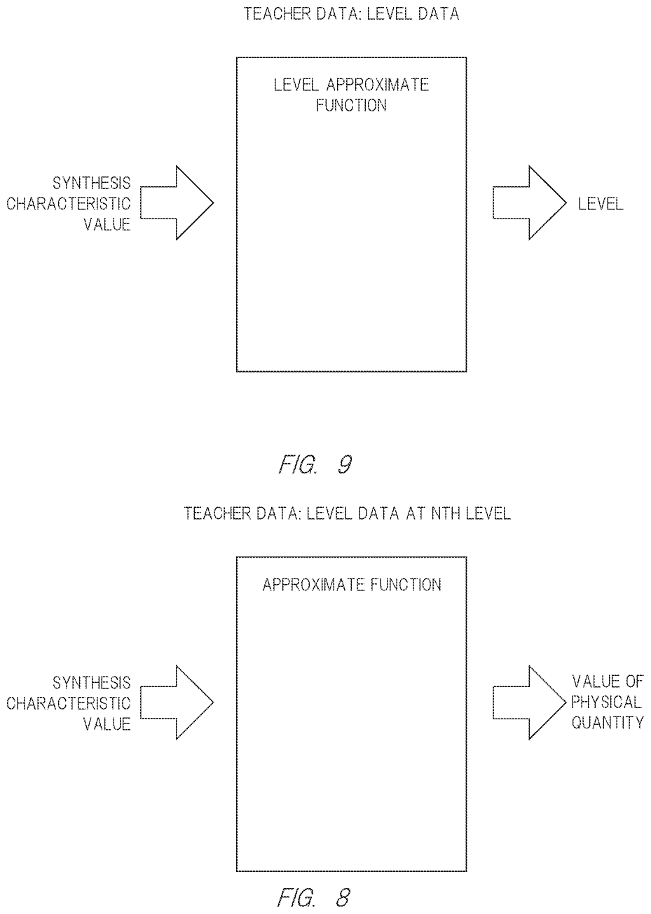

A level approximate function generating unit 702 has a function of generating a level approximate function on the basis of the level data generated by the level data generating unit 701. Namely, the level approximate function generating unit 702 has a function of generating a level approximate function that associate the synthesis characteristic value with the level to which the value of the physical quantity for a composite material belongs. Specifically, as illustrated in FIG. 8, the level approximate function generating unit 702 is configured to use the level data as teacher data to generate the level approximate function in which an input is set to the synthesis characteristic value and an output is set to the level.

Here, the “level approximate function” is defined as a function to output, when a synthesis characteristic value is inputted thereinto, a level to which a value of physical quantity according to this synthesis characteristic value belongs. Namely, the “level approximate function” is defined as a function in which in a case where a synthesis characteristic value of a composite material whose correspondence with a level of physical quantity is unknown is inputted thereinto, a level to which a value of physical quantity presumed to be realized by this composite material belongs is outputted. As described above, the level approximate function can be said to be a function used to estimate the level corresponding to the synthesis characteristic value whose correspondence with the value of the physical quantity is unknown.

A level estimating unit 704 has a function of estimating a value of physical quantity corresponding to a first composite material on the basis of a first synthesis characteristic value calculated based on a first blending ratio and first related data of the first composite material by a synthesis characteristic value calculating unit 303 and the level approximate function generated by the level approximate function generating unit 702. Namely, the level estimating unit 704 is configured so as to estimate the level to which the value of the physical quantity for the composite material, which becomes an evaluation target, belongs by using the level approximate function generated by the level approximate function generating unit 702.

An approximate function generating unit 703 has a function of generating an approximate function that associates the synthesis characteristic value with the value of the physical quantity for each of the plurality of levels. Namely, the approximate function generating unit 703 is configured to generate a different approximate function for each different level. Therefore, there are as many approximate functions generated by the approximate function generating unit 703 as the number of levels.

Specifically, as illustrated in FIG. 9, the approximate function generating unit 703 has a function of generating an approximate function for an Nth level in which level data whose level is an Nth level of the level data are used as teacher data, an input is the synthesis characteristic value, and an output is the value of the physical quantity. For example, the approximate function generating unit 703 is configured to generate an approximate function for each of a first level to the Nth level so that level data whose level is each of the first level to the Nth level of the level data are used as teacher data, an input is the synthesis characteristic value, and an output is the value of the physical quantity. Namely, in a case where the level data are classified into N pieces of levels, the approximate function generating unit 703 generates N pieces of different approximate functions.

Here, the “approximate function” is defined as a function in which when a synthesis characteristic value is inputted, a value of physical quantity according to this synthesis characteristic value is outputted. Namely, the “approximate function” is defined as a function in which in a case where a synthesis characteristic value whose correspondence with a value of physical quantity is unknown is inputted, a value of physical quantity presumed to be realized by a composite material having this synthesis characteristic value is outputted. As described above, the approximate function can be said to be a function used to estimate a value of physical quantity corresponding to a synthesis characteristic value whose correspondence with the value of the physical quantity is unknown.

A physical quantity estimating unit 705 has a function of estimating the value of the physical quantity corresponding to the composite material of the evaluation target on the basis of the approximate function for the level estimated by the level estimating unit 704.

For example, in a case where the level estimated by the level estimating unit 704 is the first level, the physical quantity estimating unit 705 uses the approximate function for the first level among the plurality of approximate functions generated by the approximate function generating unit 703 to estimate the value of the physical quantity corresponding to the composite material of the evaluation target.

### <<Operation of Physical Quantity Estimating Apparatus>>

The physical quantity estimating apparatus 100A is configured as described above. Hereinafter, an operation thereof will be described. The operation of the physical quantity estimating apparatus 100A includes a “generating operation of a level approximate function”, a “generating operation of an approximate function”, and an “estimating operation of a value of physical quantity for a composite material of an evaluation target”. For this reason, these operations will be described below.

### <<<Generating Operation of Level Approximate Function>>>

FIG. 10 is a flowchart for explaining a generating operation of a level approximate function.

In FIG. 10, the level data generating unit 701 obtains synthesis related data stored in a data storage unit 308, and generates level data from the synthesis related data on the basis of a level classification standard set in advance (S301). For example, as the level classification set in advance, a standard, in which it is set to a first level when a value of physical quantity is a predetermined threshold value or less, while it is set to a second level when the value of the physical quantity is larger than the predetermined threshold value, can be thought. Note that the level classification set in advance may be not only a standard to be classified into two types of levels, but also a standard to be classified into three or more kinds of levels. Then, the level data generated by the level data generating unit 701 are stored in the data storage unit 308.

Subsequently, the level approximate function generating unit 702 generates a level approximate function on the basis of the level data generated by the level data generating unit 701 (S302). Specifically, the level approximate function generating unit 702 generates a level approximate function in which the level data are used as teacher data, an input is a synthesis characteristic value, an output is a level. The generating operation of the level approximate function is executed in this manner.

### <<<Generating Operation of Approximate Function>>>

Next, a generating operation of an approximate function will be described.

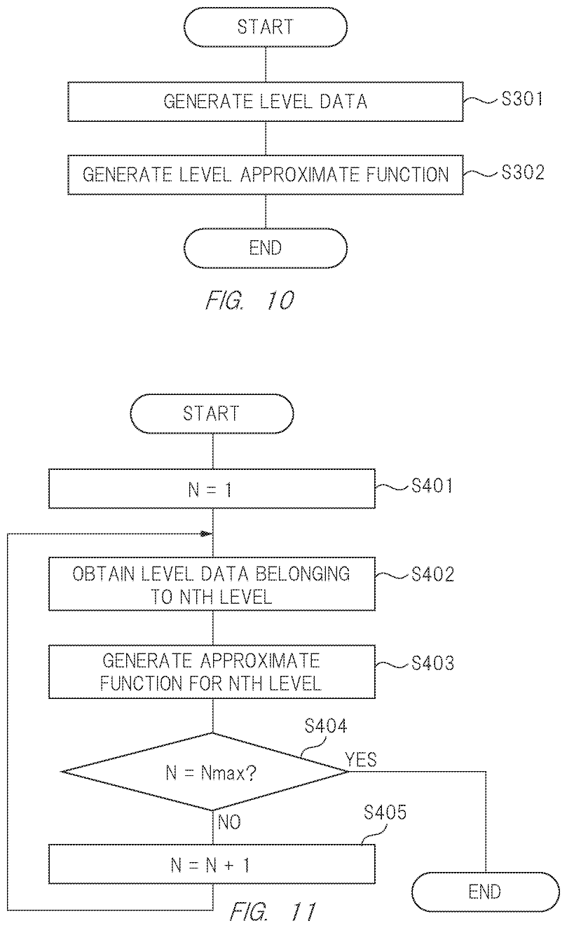

FIG. 11 is a flowchart for explaining a generating operation of an approximate function.

Here, it is assumed that level data have already been stored in the data storage unit 308. For example, it is assumed that level data at a first level to level data at an Nmax level are stored in the data storage unit 308.

The physical quantity estimating apparatus 100A first sets “N” indicating a level to N=1 (S401). Subsequently, the physical quantity estimating apparatus 100A obtains level data belonging to the Nth level from the level data stored in the data storage unit 308 (S402).

The approximate function generating unit 703 then generates an approximate function for the Nth level on the basis of the level data belonging to the Nth level (S403). Specifically, the approximate function generating unit 703 uses the level data belonging to the Nth level as teacher data to generate the approximate function for the Nth level in which an input is a synthesis characteristic value and an output is a value of physical quantity. The generated approximate function for the Nth level is stored in the data storage unit 308.

The physical quantity estimating apparatus 100A then determines whether the “N” indicating the level is an Nmax or not (S404). At this time, in a case where it is determined that the “N” indicating the level is the Nmax, the physical quantity estimating apparatus 100A terminates the generating operation of the approximate function. On the other hand, in a case where it is determined that the “N” indicating the level is not the Nmax, the physical quantity estimating apparatus 100A plugs in “N=N+1” (S405), and executes a generating operation of an approximate function for an (N+1)th level. As described above, it is possible to generate the approximate function for the first level to the approximate function for the Nmax level.

### <<<Estimating Operation of Value of Physical Quantity Corresponding to Composite Material of Evaluation Target>>>

Next, an operation of estimating a value of physical quantity corresponding to a composite material of an evaluation target will be described.

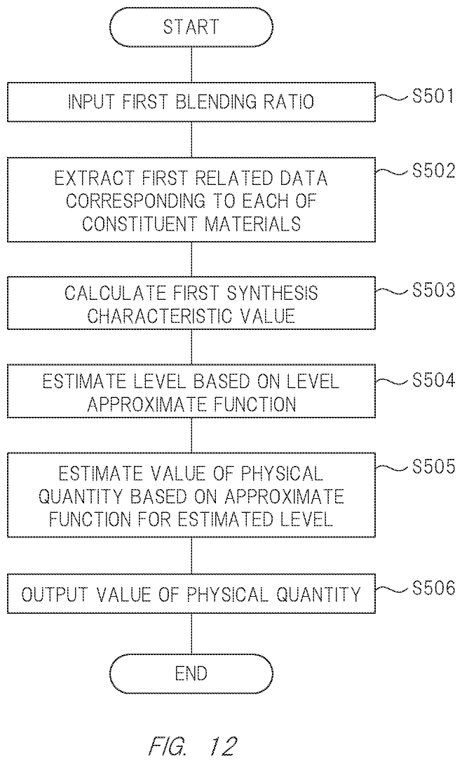

FIG. 12 is a flowchart for explaining an operation of estimating a value of physical quantity corresponding to a composite material of an evaluation target. Here, it is assumed that a level approximate function and an approximate function for each of a first level to an Nmax level have already been stored in the data storage unit 308.

In FIG. 12, the input unit 301 first inputs a first blending ratio of constituent materials contained in a first composite material whose correspondence with a value of physical quantity is unknown and that becomes an evaluation target (S501).

Next, the related data extracting unit 302 extracts first related data corresponding to each of the constituent materials contained in the first composite material from a plurality of related data stored in the data storage unit 308 (S502).

Subsequently, the synthesis characteristic value calculating unit 303 calculates a first synthesis characteristic value of the first composite material by executing an operation of synthesizing characteristic values contained in the first related data extracted by the related data extracting unit 302 on the basis of the first blending ratio inputted by the input unit 301 (S503).

Subsequently, the level estimating unit 704 estimates a level corresponding to the first synthesis characteristic value on the basis of the first synthesis characteristic value calculated by the synthesis characteristic value calculating unit 303 and the level approximate function stored in the data storage unit 308 (S504).

Subsequently, the physical quantity estimating unit 705 estimates the value of the physical quantity corresponding to the first composite material of an evaluation target on the basis of the first synthesis characteristic value calculated by the synthesis characteristic value calculating unit 303 and the approximate function for the level estimated by the level estimating unit 704 (S505). The output unit 307 then outputs the value of the physical quantity estimated by the physical quantity estimating unit 705 (S506).

As described above, according to the physical quantity estimating apparatus 100A, it is possible to output the value of the physical quantity, which is highly likely to be realized by the first composite material whose correspondence with the value of the physical quantity is unknown and that becomes the evaluation target.

### <<Feature According to Embodiment>>

Subsequently, a feature according to the present embodiment will be described.

A feature according to the present embodiment is that a value of physical quantity corresponding to a composite material is divided into a plurality of levels and a different approximate function is generated for each of the plurality of divided levels. This makes it possible to estimate a value of physical quantity for a composite material whose correspondence with the value of the physical quantity is unknown with high accuracy. Hereinafter, this point will be described.

For example, it is assumed that a numerical range of a value of physical quantity corresponding to a synthesis characteristic value of a composite material is “0” to “1000”. In this case, it is conceivable to use synthesis related data belonging to the numerical range of “0” to “1000” as teacher data to obtain a single approximate function in which an input is the synthesis characteristic value and an output is the value of the physical quantity.

However, since the numerical range of the value of the physical quantity is wide, it is difficult even for machine learning to presume the value of the physical quantity with high accuracy in all the of the numerical range by the single approximate function. Namely, in the single approximate function for the physical quantity, it is difficult to estimate the value of the physical quantity over the wide numerical range with high accuracy even by using the machine learning.

Therefore, in the present embodiment, the numerical range of the value of the physical quantity is divided, and the optimum approximate function is set for each of narrow divided numerical ranges. For example, the numerical range of “0” to “1000” described above is divided into a first numerical range of “0” to “300” and a second numerical range of “301” to “1000”, the first numerical range is set as a first level and the second numerical range is set as a second level. As a result, in the present embodiment, level data at the first level in which a numerical range of the value of the physical quantity for the synthesis characteristic value is the first numerical range and level data at the second level in which the numerical range of the value of the physical quantity for the synthesis characteristic value is the second numerical range are generated.

Then, the level data belonging to the first level are used as teacher data to obtain an approximate function for the first level in which an input is the synthesis characteristic value and an output is the value of the physical quantity. Similarly, the level data belonging to the second level are used as teacher data to obtain an approximate function for the second level in which an input is the synthesis characteristic value and an output is the value of the physical quantity.

As a result, since the numerical range (“0” to “300”) of the value of the physical quantity contained in the level data used for the teacher data becomes narrow, it becomes possible to obtain the approximate function for the first level by which it is possible to estimate the value of the physical quantity with high accuracy. Similarly, since the numerical range (“301” to “1000”) of the value of the physical quantity contained in the level data used for the teacher data becomes narrow, it becomes possible to obtain the approximate function for the second level by which it is possible to estimate the value of the physical quantity with high accuracy.

As described above, the feature according to the present embodiment is based on the finding that a high accurate approximate function can be obtained by limiting the numerical range of the level data used for the teacher data. In order to embody this finding, in the present embodiment, the synthesis related data are divided into the plurality of levels. Here, the important point is to accurately estimate a level corresponding to a synthesis characteristic value whose correspondence with a value of physical quantity is unknown. This is because when a level for a synthesis characteristic value whose correspondence with the value of the physical quantity is unknown is estimated as a different level from a level to which a synthesis characteristic value actually belong, an approximate function for the different level, which is not an approximate function to be used to estimate the value of the physical quantity with high accuracy, is used, and as a result, the value of the physical quantity cannot be estimated with high accuracy. Therefore, in order to accurately estimate the level corresponding to the synthesis characteristic value whose correspondence with the value of the physical quantity is unknown, in the present embodiment, level data that associate synthesis related data with a level are generated. Then, by using the level data as teacher data, a level approximate function in which an input is the synthesis characteristic value and an output is the level is obtained. By using the level approximate function in this manner, it is possible to accurately estimate the level corresponding to the synthesis characteristic value whose correspondence with the value of the physical quantity is unknown.

Namely, in the present embodiment, by using the level approximate function for estimating the level and the approximate function generated for each level, it is possible to estimate the value of the physical quantity corresponding to the composite material whose correspondence with the value of the physical quantity is unknown with high accuracy.

### <<Verification of Effect>>

Hereinafter, a verification result in which according to the present embodiment, it is possible to estimate the value of the physical quantity corresponding to the composite material whose correspondence with the value of the physical quantity is unknown with high accuracy will be described.

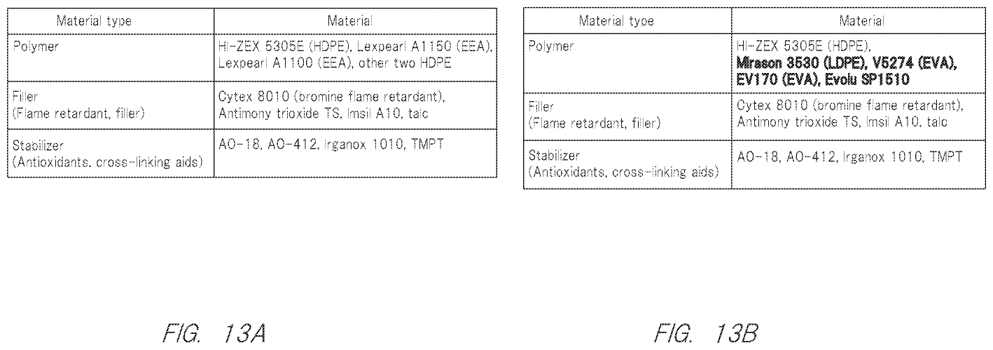

FIG. 13A is a table illustrating one example of a composite material used when generating an approximate function. Namely, the approximate function is generated by machine learning using a composite material shown in FIG. 13A as teacher data. The generated approximate function is then used to estimate a value of physical quantity for a composite material of an evaluation target shown in FIG. 13B, for example.

Here, a material that is not contained in the composite material shown in FIG. 13A and used as the teacher data is contained in the composite material shown in FIG. 13B. Specifically, a polymer indicated by a bold face in FIG. 13B is the material that is not contained in the composite material shown in FIG. 13A.

First, results of estimating a value of physical quantity for the composite material of the evaluation target shown in FIG. 13B by using the related technique will be described. Namely, in the related technique, when a material name of each of the constituent materials constituting the composite material and a blending ratio of the constituent materials are inputted, a value of physical quantity for the composite material is estimated on the basis of an approximate function that outputs the value of the physical quantity for this composite material.

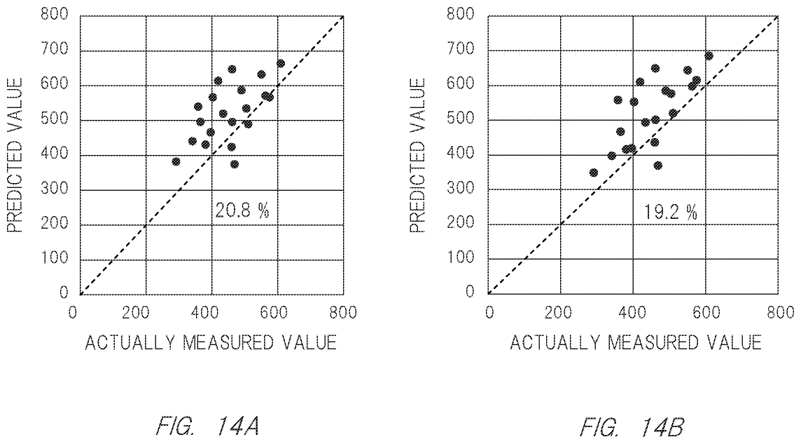

FIG. 14A is a graph illustrating results of estimating a value of physical quantity for the composite material of the evaluation target shown in FIG. 13B by using the related technique.

In FIG. 14A, a horizontal axis thereof indicates actually measured values, while a vertical axis thereof indicates predicted values. As illustrated in FIG. 14A, dispersion of data is “20.8%”.

Subsequently, results of estimating a value of physical quantity for the composite material of the evaluation target shown in FIG. 13B by using the technical idea according to the present embodiment will be described. Namely, in the technical idea according to the present embodiment, a value of physical quantity for a composite material is estimated by using an approximate function generated on the basis of a characteristic value (a numerical value) of each of constituent materials constituting the composite material and a blending ratio of the constituent materials.

FIG. 14B is a graph illustrating results of estimating a value of physical quantity for the composite material of the evaluation target shown in FIG. 13B by using the technical idea according to the present embodiment.

In FIG. 14B, a horizontal axis thereof indicates actually measured values, while a vertical axis thereof indicates predicted values. As illustrated in FIG. 14B, dispersion of data is “19.2%”.

At this time, it is considered that the smaller the dispersion of the data is, the closer the predicted values are to the actually measured values. According to the technical idea according to the present embodiment, it can be seen that estimation accuracy of the value of the physical quantity (that is, the dispersion of the data) is improved by “1.6%” as compared with the related technique. Therefore, from the verification results of FIG. 14A and

FIG. 14B, according to the technical idea by the present embodiment, it can be seen that it is supported that it is possible to estimate the value of the physical quantity for the composite material whose correspondence with the value of the physical quantity is unknown with high accuracy. Namely, from the verification results of FIG. 14A and FIG. 14B, it can be seen that the technical idea according to the present embodiment is a very useful technical idea in that it is possible to estimate the value of the physical quantity for the composite material whose correspondence with the value of the physical quantity is unknown with high accuracy.

As described above, the invention made by the inventors of the present application has been described specifically on the basis of the embodiments. However, the present invention is not limited to the first to third embodiments described above, and it goes without saying that the present invention may be modified into various forms without departing from the substance thereof.

The embodiment includes the following forms.

### APPENDIX 1

An approximate function generating apparatus that is a component of a physical quantity estimating system for estimating physical quantity, the approximate function generating apparatus including:

a related data storage unit configured to store a plurality of related data, a characteristic value of each of a plurality of different materials being associated with a value of physical quantity for the corresponding material in each of the plurality of related data;

a blending ratio input unit configured to input a blending ratio of constituent materials of a composite material, the composite material containing two or more materials included in the plurality of different materials as the constituent materials, a value of corresponding physical quantity for the composite material being known;

a related data extracting unit configured to extract related data corresponding to each of the constituent materials from the plurality of related data;

a synthesis characteristic value calculating unit configured to calculate a synthesis characteristic value of the composite material by executing an operation of synthesizing the characteristic values included in the corresponding related data of the respective constituent materials on the basis of the blending ratio;

a synthesis related data generating unit configured to generate synthesis related data in which the synthesis characteristic value is associated with the value of the physical quantity for the composite material; and

an approximate function generating unit configured to generate, when a first synthesis characteristic value of a first composite material whose value of the corresponding physical quantity is unknown is inputted, an approximate function of outputting a value of the physical quantity for the first composite material on a basis of the synthesis related data.

### APPENDIX 2

A physical quantity estimating apparatus that is a component of a physical quantity estimating system for estimating physical quantity, the physical quantity estimating apparatus including:

a blending ratio input unit configured to input a first blending ratio of constituent materials contained in a first composite material, physical quantity corresponding to the first composite material is unknown;

a related data extracting unit configured to extract first related data corresponding to each of the constituent materials from a plurality of related data in which a characteristic value of each of a plurality of different materials is associated with a value of physical quantity for the material;

a synthesis characteristic value calculating unit configured to calculate a first synthesis characteristic value of the first composite material on a basis of the first blending ratio and the first related data;

a physical quantity estimating unit configured to estimate, when the first synthesis characteristic value is inputted, the value of the physical quantity corresponding to the first blending ratio on a basis of an approximate function of outputting the value of the physical quantity for the first composite material; and

an output unit configured to output the value of the physical quantity estimated by the physical quantity estimating unit.

1. A physical quantity estimating system for estimating a value of physical quantity for a composite material, the composite material containing two or more materials included in a plurality of different materials as constituent materials, the physical quantity estimating system comprising:
an approximate function generating unit configured to generate, when a first synthesis characteristic value of a first composite material whose value of physical quantity is unknown is inputted, an approximate function of outputting the value of the physical quantity for the first composite material;
a synthesis characteristic value calculating unit configured to calculate the first synthesis characteristic value of the first composite material on a basis of a first blending ratio of constituent materials contained in the first composite material and first related data corresponding to each of the constituent materials contained in the first composite material; and
a physical quantity estimating unit configured to estimate the value of the physical quantity for the first composite material on a basis of the first synthesis characteristic value and the approximate function.

2. The physical quantity estimating system according to claim 1,
wherein the first related data are data in which a characteristic value of each of the constituent materials contained in the first composite material is associated with a value of physical quantity for each of the constituent materials contained in the first composite material.

3. A physical quantity estimating system comprising:
a related data storage unit configured to store a plurality of related data, a characteristic value of each of a plurality of different materials being associated with a value of physical quantity for the corresponding material in each of the plurality of related data;
a blending ratio input unit configured to input a blending ratio of constituent materials of a composite material, the composite material containing two or more materials included in the plurality of different materials as the constituent materials, a value of corresponding physical quantity for the composite material being known;
a related data extracting unit configured to extract related data corresponding to each of the constituent materials from the plurality of related data;
a synthesis characteristic value calculating unit configured to calculate a synthesis characteristic value of the composite material by executing an operation of synthesizing the characteristic values included in the corresponding related data of the respective constituent materials on a basis of the blending ratio;
a synthesis related data generating unit configured to generate synthesis related data in which the synthesis characteristic value is associated with the value of the physical quantity for the composite material; and
an approximate function generating unit configured to generate, when a first synthesis characteristic value of a first composite material whose value of the corresponding physical quantity is unknown is inputted, an approximate function of outputting a value of the physical quantity for the first composite material on a basis of the synthesis related data,
wherein the blending ratio input unit is configured to input a first blending ratio of the constituent materials contained in the first composite material,
wherein the related data extracting unit is configured to extract first related data corresponding to each of the constituent materials contained in the first composite material from the plurality of related data,
wherein the synthesis characteristic value calculating unit is configured to calculate the first synthesis characteristic value of the first composite material on a basis of the first blending ratio and the first related data, and
wherein the physical quantity estimating system further comprises:
a physical quantity estimating unit configured to estimate a value of the physical quantity corresponding to the first blending ratio on a basis of the first synthesis characteristic value and the approximate function; and
an output unit configured to output the value of the physical quantity estimated by the physical quantity estimating unit.

4. The physical quantity estimating system according to claim 3,
wherein the approximate function generating unit is configured to generate the approximate function in which the synthesis related data are used as teacher data, an input is the synthesis characteristic value, and an output is the value of the physical quantity.

5. The physical quantity estimating system according to claim 3,
wherein the constituent materials of the composite material include a resin.

6. The physical quantity estimating system according to claim 5,
wherein the characteristic value of the resin includes any of thermal characteristics of the resin, mechanical characteristics of the resin, and a physical property of the resin.

7. The physical quantity estimating system according to claim 6,
wherein the thermal characteristics of the resin include any of heat of fusion of the resin, a melt flow rate of the resin, and viscosity of the resin.

8. The physical quantity estimating system according to claim 6,
wherein the physical property of the resin includes specific gravity of the resin.

9. The physical quantity estimating system according to claim 3,
wherein the synthesis characteristic value includes either synthesis heat of fusion of the composite material or a synthesis melt flow rate of the composite material.

10. The physical quantity estimating system according to claim 5,
wherein the constituent materials of the composite material further contain additives,
wherein the synthesis characteristic value calculating unit is further configured to calculate an average inter-filler distance of the additives, volume fraction of the additives or a BET specific surface area on a basis of the characteristic value of the additives, and
wherein the synthesis characteristic value further includes the average inter-filler distance of the additives or the volume fraction of the additives.

11. The physical quantity estimating system according to claim 5,
wherein the constituent materials of the composite material further contain antioxidants and cross-linking aids,
wherein the synthesis characteristic value calculating unit is further configured to calculate a reaction mole number of a primary reactive group of the antioxidants, a reaction mole number of a secondary reactive group of the antioxidants, and a reaction mole number of the cross-linking aids on a basis of a characteristic value of the antioxidants and a characteristic value of the cross-linking aids, and
wherein the synthesis characteristic value further includes the reaction mole number of the primary reactive group of the antioxidants, the reaction mole number of the secondary reactive group of the antioxidants, and the reaction mole number of the cross-linking aids.

12. The physical quantity estimating system according to claim 3,
wherein the constituent materials of the composite material contain crosslinked resin,
wherein the blending ratio input unit is further configured to input a radiation dose for crosslinking the resin, and
wherein the approximate function generating unit is configured to generate the approximate function on the basis of the synthesis related data and the radiation dose.

13. The physical quantity estimating system according to claim 3,
wherein the physical quantity for the composite material is an elongation characteristic.

14. A physical quantity estimating method of estimating a value of physical quantity for a composite material, the composite material containing two or more materials included in a plurality of different materials as constituent materials, the physical quantity estimating method comprising:
an approximate function generating step configured to generate, when a first synthesis characteristic value of a first composite material whose value of physical quantity is unknown is inputted, an approximate function of outputting the value of the physical quantity for the first composite material;
a synthesis characteristic value calculating step configured to calculate the first synthesis characteristic value of the first composite material on a basis of a first blending ratio of constituent materials contained in the first composite material and first related data corresponding to each of the constituent materials contained in the first composite material; and
a physical quantity estimating step configured to estimate the value of the physical quantity for the first composite material on a basis of the first synthesis characteristic value and the approximate function.

15. The physical quantity estimating method according to claim 14,
wherein the first related data are data in which a characteristic value of each of the constituent materials contained in the first composite material is associated with a value of physical quantity for each of the constituent materials contained in the first composite material.

16. A physical quantity estimating method comprising:
a related data storage step configured to store a plurality of related data in a related data storage unit, a characteristic value of each of a plurality of different materials being associated with a value of physical quantity for the corresponding material in each of the plurality of related data;
a blending ratio input step configured to input a blending ratio of constituent materials of a composite material, the composite material containing two or more materials included in the plurality of different materials as the constituent materials, a value of corresponding physical quantity for the composite material being known;
a related data extracting step configured to extract related data corresponding to each of the constituent materials from the plurality of related data;
a synthesis characteristic value calculating step configured to calculate a synthesis characteristic value of the composite material by executing an operation of synthesizing the characteristic values included in the corresponding related data of the respective constituent materials on the basis of the blending ratio;
a synthesis related data generating step configured to generate synthesis related data in which the synthesis characteristic value is associated with the value of the physical quantity for the composite material; and
an approximate function generating unit configured to generate, when a first synthesis characteristic value of a first composite material whose value of the corresponding physical quantity is unknown is inputted, an approximate function of outputting a value of the physical quantity for the first composite material on a basis of the synthesis related data,
wherein the blending ratio input step is configured to input a first blending ratio of the constituent materials contained in the first composite material,
wherein the related data extracting step is configured to extract first related data corresponding to each of the constituent materials contained in the first composite material from the plurality of related data,
wherein the synthesis characteristic value calculating step is configured to calculate the first synthesis characteristic value of the first composite material on a basis of the first blending ratio and the first related data, and
wherein the physical quantity estimating method further comprises:
a physical quantity estimating step configured to estimate a value of the physical quantity corresponding to the first blending ratio on a basis of the first synthesis characteristic value and the approximate function; and
an output step configured to output the value of the physical quantity estimated in the physical quantity estimating step.
